(19) United States
(12) Patent Application Publication
(10) Pub. No.: US 2016/0242834 A1
(43) Pub. Date: Aug. 25, 2016

(54) DISPENSING DEVICE WITH SPRING TONGUES FOR CEMENT CARTRIDGES

(71) Applicant: Heraeus Medical GmbH, Wehrheim (DE)

(72) Inventor: Sebastian VOGT, Erfurt (DE)

(73) Assignee: Heraeus Medical GmbH, Wehrheim (DE)

(21) Appl. No.: 15/042,206

(22) Filed: Feb. 12, 2016

(30) Foreign Application Priority Data
Feb. 20, 2015 (DE) 10 2015 102 463.2

(51) Int. Cl. A61B 17/88

(52) U.S. Cl. CPC A61B 17/8825; A61B 17/8822; A61B 2017/00367

(57) ABSTRACT

A dispensing device comprises at least one manually actuatable operating element, an adapter for a cement cartridge, and a clamping rod that can be propelled in the direction of the adapter, whereby at least one propulsion body is arranged to be touching against the clamping rod, whereby the at least one propulsion body comprises multiple elastic spring tongues. The spring tongues each comprise a cutting edge, whereby the spring tongues touch against the clamping rod by means of the cutting edge, and whereby the spring tongues of the propulsion body are inclined appropriately with respect to the clamping rod such that, upon a motion of the propulsion body in the propulsion direction of the clamping rod, the cutting edges of the propulsion body engage the clamping rod. A spring element pushes with an elastic force, at least for part of the time, onto the at least one propulsion body opposite to the propulsion direction of the clamping rod, whereby a force can be exerted on the at least one propulsion body in the propulsion direction of the clamping rod by means of the operating element, whereby a securing element, against which the propulsion body is supported such as to be mobile, touches against the clamping rod, whereby the securing element comprises multiple elastic spring tongues. The spring tongues each comprise a cutting edge, whereby the spring tongues touch against the clamping rod by means of the cutting edge, and whereby the spring tongues are inclined appropriately with respect to the clamping rod such that, upon a motion of the clamping rod opposite to the propulsion direction, the cutting edges of the securing element engage the clamping rod.

The invention relates to a dispensing device for cement cartridges for polymethylmethacrylate bone cements comprising at least one manually actuable operating element, an adapter for a cement cartridge, and a clamping rod that can be propelled in the direction of the adapter.

Accordingly, the subject matter of the invention is a manually driven device for dispensing polymethylmethacrylate bone cement dough from cartridges of vacuum cementing systems. The devices designed for single use only and should be recyclable for heat production after use (by incineration).

Articular endoprostheses are used commonly in orthopaedics and trauma surgery to replace human joints that have been damaged by disease, accident or wear. In this context, permanent mechanical fixation of articular endoprostheses is effected by mechanical clamping (press-fit) or by cementing using polymethylmethacrylate bone cements (PMMA bone cements).

PMMA bone cements consist of a liquid monomer component and a powder component. The monomer component generally contains the monomer, methylmethacrylate, and an activator (N,N-dimethyl-p-toluidine) dissolved therein. The powder component, also called bone cement powder, comprises one or more polymers that are produced through polymerisation, preferably suspension polymerisation, based on methylmethacrylate and co-monomers, such as styrene, methylacrylate or similar monomers, often a radiopaquer, and the initiator, dibenzoylperoxide. The powder component is mixed with the liquid monomer component before application of the cement During the mixing process, swelling of the polymers of the powder component in the methylmethacrylate generates a dough that can be shaped plastically and is the actual bone cement. In this context, the activator, N,N-dimethyl-p-toluidine, reacts with dibenzoylperoxide forming radicals in the process. The radicals thus formed trigger the radical polymerisation of the methylmethacrylate. Upon advancing polymerisation of the methylmethacrylate, the viscosity of the cement dough increases until the cement dough solidifies. The solidified polymethylmethacrylate bone cement is mechanically stable and can permanently attach articular endoprostheses in the bone tissue of the patients.

The components of polymethylmethacrylate bone cements (PMMA bone cements) can be mixed with spatulas in suitable mixing cups. One disadvantage of said procedure is that air inclusions may be present in the cement dough thus formed and can cause destabilisation of the cured bone cement later on. For this reason, it is preferred to mix bone cement powder and monomer liquid in vacuum mixing systems, since mixing in a vacuum removes air inclusions from the cement dough to a large extent and thus achieves optimal cement quality. Bone cements mixed in a vacuum have clearly reduced porosity and thus show improved mechanical properties. A large number of vacuum cementing systems have been disclosed of which the following shall be listed for exemplary purposes: U.S. Pat. No. 6,033,105 A, U.S. Pat. No. 5,624,184 A, U.S. Pat. No. 4,671,263 A, U.S. Pat. No. 4,973,168 A, U.S. Pat. No. 5,100,241 A, WO 99/67015 A1, EP 1 020 167 B1, U.S. Pat. No. 5,586,821 A, EP 1 016 452 A2, DE 36 40 279 C2, WO 94/26403 A1, EP 1 005 901 A2, U.S. Pat. No. 5,344,232 A.

Cementing systems, in which both the cement powder and the monomer liquid are already packed in separate compartments of the mixing systems and are mixed with each other in the cementing system only right before application of the cement, are a development of cementing technology. Such full-prepacked mixing systems have been proposed in the patents, EP 0 692 229 B1, DE 10 2009 031 178 B3, EP 0 875 456 B3, U.S. Pat. No. 6,709,149 B1, and EP 1 140 234 B1 as well as U.S. Pat. No. 5,588,745 A.

Used in vacuum cementing systems, it is necessary to expel the cement dough from the cement cartridges by moving a plunger in order to apply the cement dough. Manual dispensing devices have been developed for this purpose.

EP 0 326 551 A1 prescribed an interesting lever system for manually driven devices. The rationale of said lever system is based on utilising a lever parallelogram to make optimally use of the force of the strongest fingers of the human hand, namely the index finger and middle finger, which is in contrast to a simple rocker lever.

The simplest dispensing devices are based on clamping rods having a tiltable small metal plate arranged on them that gets tilted by an asymmetrically engaging spring and thus clamps the clamping rod. The tiltable small metal plate is pressed in propulsion direction by a lever upon manual actuation, whereby the clamping small metal plate takes the clamping rod along with it. Subsequently, a spring pushes the small metal plate back into its starting position. This process is repeated until the clamping rod has pressed the feed plunger of the vacuum cementing system sufficiently far in the direction of the cartridge head such that the desired amount of bone cement is pressed from the cartridge.

These devices are disadvantageous in that a retrograde motion due to the small metal plate sliding on the clamping rod is always possible during the clamping process. Due to the small metal plate sliding backwards, the user needs significantly more hand motions for extruding the bone cement dough than actually needed.

A further development consists of dispensing devices that use gear racks instead of simple clamping rods. One pertinent example is the dispensing device according to FIG. 28 of patent application US 2013 090 661 A1. It is an advantage of said devices that retrograde motion is basically excluded. However, the complex, laborious mechanics rendering the utilisation of said device for single use only questionable due to the relatively high production costs is disadvantageous.

It is the object of the invention to overcome the disadvantages of the prior art. Specifically, a dispensing device that is easy to manufacture is to be provided, by means of which a polymethylmethacrylate bone cement dough can be expelled manually from cement cartridges of vacuum cementing systems. The manually driven device shall be suitable and intended for single use only. The dispensing device is to consist largely of inexpensive plastic parts, which can be manufactured by plastics injection moulding, and of few metal elements. Specifically, the dispensing device is to be designed to include a clamping rod made of plastics. A clamping rod made of metal, in particular of steel, is to be avoided Due to the device being designed to be made almost completely of plastics, the device can be recycled for heat production after single-use (by incineration in a waste incineration plant) without any major amounts of metal being produced and/or being lost.

During manual actuation of the dispensing device, a retrograde motion of the feed plunger of the cartridge during the extrusion process due to elastic restoring forces of the plastic cartridge and the cement dough is to be prevented to the extent possible. The dispensing device to be developed is to enable just a single dispensation of polymethylmethacrylate bone cement dough. Reuse and the ensuing need for re-sterilisation of the dispensing device shall be excluded by the design.

The objects of the invention are met by a dispensing device for cement cartridges for polymethylmethacrylate bone cements comprising at least one manually actuatable operating element, an adapter for a cement cartridge, and a clamping rod that can be propelled in the direction of the adapter, whereby at least one propulsion body is arranged to touch against the clamping rod and can be shifted in axial direction with respect to the axis of the clamping rod,

whereby the at least one propulsion body comprises multiple elastic spring tongues, whereby the spring tongues each comprise a cutting edge, whereby the spring tongues touch against the clamping rod by means of the cutting edge, whereby the cutting edges of the spring tongues or the entire spring tongues are harder than the clamping rod, and whereby the spring tongues of the propulsion body are inclined appropriately with respect to the clamping rod such that, upon a motion of the propulsion body in propulsion direction of the clamping rod, the cutting edges of the propulsion body engage the clamping rod, and such that, upon a motion of the propulsion body opposite to the propulsion direction of the clamping rod, the spring tongues of the propulsion body can be elastically deformed appropriately such that the cutting edges slide over the clamping rod,
whereby a spring element acts on the at least one propulsion body, at least for part of the time, by an elastic force opposite to the propulsion direction of the clamping rod,
whereby a force can be exerted on the at least one propulsion body in the propulsion direction of the clamping rod by means of the operating element, whereby the propulsion body and the clamping rod can be propelled forward relative to the adapter against the elastic force of the spring element by means of said force, whereby a securing element, against which the propulsion body is supported such as to be mobile, touches against the clamping rod,
whereby the securing element comprises multiple elastic spring tongues, whereby the spring tongues each comprise a cutting edge, whereby the spring tongues touch against the clamping rod by means of the cutting edge, whereby at least the cutting edges of the spring tongues or the entire spring tongues are harder than the clamping rod, and whereby the spring tongues are inclined appropriately with respect to the clamping rod such that, upon a motion of the clamping rod opposite to the propulsion direction, the cutting edges of the securing element engage the clamping rod, and such that, upon a motion of the clamping rod in the propulsion direction, the spring tongues of the securing element can be elastically deformed appropriately such that the cutting edges slide over the clamping rod.

In the scope of the present invention, it being possible to propel the clamping rod in the direction of the adapter also means that the clamping rod can be propelled through the adapter even if the centre of gravity of the clamping rod then actually moves away from the adapter towards the end of the dispensing process. The axis of the clamping rod shall be understood to be the geometric axis of the clamping rod, rather than a rotation axis.

The invention preferably provides the dispensing device to be suitable for manual expulsion of cement cartridges filled with polymethylmethacrylate bone cement or with starting components of PMMA bone cement. It is particularly preferable to hold the dispensing device with one hand and to operate it with the same hand. For expulsion, the cement cartridge must be connected to the adapter of the dispensing device.

According to the invention, the cutting edges can be formed by a plane edge, a wavy edge or a sawtooth-like edge or by tips. In this context, the cutting edge does not have to actually cut into the clamping rod, but it can simply be impressed into it or engage the clamping rod by any other means in that the clamping rod is plastically deformed by the cutting edge.

The cutting edges engaging the clamping rod shall be understood to mean that the cutting edges cut into the clamping rod, are impressed into the clamping rod or penetrate in other ways into the surface of the clamping rod. This is associated with a plastic deformation of the clamping rod, particularly at the surface thereof.

The spring element preferably acts, in particular the spring element pushes, on the at least one propulsion body in any position of the at least one propulsion body, by the elastic force acting opposite to the propulsion direction of the clamping rod. If multiple propelling bodies are provided, the spring element can be provided to comprise multiple individual springs, which act on the various propelling bodies either individually or in groups.

Presently, the axial direction shall always be the direction along which the axis of the clamping rod extends and can be moved.

It is theoretically feasible to use, instead of a clamping rod that can be propelled in the direction of the adapter, a clamping rod that can be propelled in working direction if the force arising from the propulsion of the clamping rod is redirected by joints, gear racks or the like, and if the working direction is changed thereby, and if the adapter for connection of the cement cartridge is situated in this location, and the clamping rod can therefore no longer be propelled geometrically in the direction of the adapter. This shall be understood as an equivalent design with the same working principle.

The invention also proposes that the at least one propulsion body is linearly mobile in axial direction with respect to the clamping rod and with respect to the adapter and/or that the securing element is connected to the dispensing device such as to be immobile with respect to the adapter.

This results in a particularly easy and inexpensively implemented design of the dispensing device.

Moreover, the invention can provide the cutting edges of the spring tongues of the securing element to appropriately engage the clamping rod during a motion of the clamping rod opposite to the propulsion direction such that any further motion of the clamping rod opposite to the propulsion direction is prevented.

This ensures that the clamping rod is prevented from being pushed backwards. This may be caused by elastic deformation of the cement cartridge during the expulsion process or by gas being compressed in the cement cartridge and thus a force being exerted on the clamping rod by the cement cartridge or its content, whereby the force acts opposite to the propulsion direction of the clamping rod even if the clamping rod is no longer being propelled to or through the adapter. Moreover, this prevents the propelled feed plunger to move backwards in the cement cartridge.

According to a preferred refinement, the invention can provide the cutting edges of the spring tongues of the at least one propulsion body to engage the clamping rod appropriately such that the clamping rod moves along with the at least one propulsion body in propulsion direction.

As a result, the clamping rod can be effectively propelled by the operating element by means of the propulsion body.

Moreover, the invention can just as well provide the elastic spring tongues of the at least one propulsion body and of the securing element to be inclined appropriately such that the cutting edges are oriented in the propulsion direction and the further parts of the spring tongues extend from the cutting edges opposite to the propulsion direction, whereby the spring tongues are preferably inclined at an angle between 88° and 20° with respect to the axis of the clamping rod, particularly preferably are inclined at an angle between 80° and 70° with respect to the axis of the clamping rod.

As a result, upon a suitable motion of the clamping rod with respect to the securing element or upon a suitable motion of the propulsion body, the spring tongues engage the clamping rod, i.e. clamp the clamping rod since the spring tongues can be elastically deformed in this direction either not at all or only with difficulty, and, upon an opposite direction of motion, the cutting edges slide over the clamping rod due to an elastic deformation of the spring tongues, i.e. do not impede the motion of the clamping rod.

Preferred dispensing devices can also be characterised in that the elastic spring tongues of the at least one propulsion body and of the securing element touch against the clamping rod appropriately such that the cutting edges are pushed onto the clamping rod by the elastic force of the elastically deformed spring tongues.

This ensures that the cutting edges of the spring tongues always contact the clamping rod and can engage the surface of the clamping rod if a suitable motion proceeds.

Moreover, the invention proposes the manually actuatable operating element to be a manually actuatable rocker lever, whereby the rocker lever is supported with respect to the clamping rod such that it can be rotated such that, upon actuation of the rocker lever, one end of the rocker lever pushes onto the at least one axially shiftable propulsion body in the propulsion direction of the clamping rod.

As a result, a lever force can be used to push the propulsion body, and thus the clamping rod, forward by a large force. It is preferred in this context that the manually operable part of the rocker lever is farther away from the pivot point than the part of the rocker lever that acts on the propulsion body, preferably is at least twice as far away. This enables a leverage of the force. If no rocker lever is used as operating element, a different transmission of force must be found for the operating element in order to be able to expel viscous PMMA cements from the cement cartridge. According to the invention, a transmission of force of at least 2:1 is preferred with at least 4:1 being particularly preferred. This means that a full stroke of the operating element is associated with the clamping rod lifting only by one half of said stroke and/or one quarter of said stroke.

Presently, a rocker lever supported as in a bearing such as to be rotatable shall be understood to be a rocker lever that can be rotated by several degrees with respect to the clamping rod. Preferably, the rocker lever can be rotated by 20° to 70° with respect to the clamping rod.

The rocker lever is preferably designed as a lever system in accordance with EP 0 326 551 A1. Said lever system uses a manually-actuated lever parallelogram that enables forceful actuation of the lever by the index finger or middle finger.

Preferred dispensing devices can be provided appropriately such that the elastic spring tongues are arranged on the internal side of at least one ring-shaped spring platelet, whereby the at least one spring platelet surrounds the clamping rod completely or by more than 50%.

By this means, the design can be simplified even more since the ring-shaped spring platelet can be incorporated in the propulsion body and/or the securing element as a uniform component. Moreover, the spring platelets can be manufactured inexpensively and/or are commercially available inexpensively.

The invention also proposes that the at least one propulsion body touching against the clamping rod and the clamping rod jointly form at least one cavity and/or that the securing element touching against the clamping rod and the clamping rod jointly form at least one cavity, whereby the spring tongues are arranged, in particular the at least one ring-shaped spring platelet is arranged, in the cavity or cavities.

By this means, the complexity of the assembly of the dispensing device can be simplified without any detrimental effect on the functionality of the dispensing device.

In this context, the invention can provide the cavity formed by the propulsion body and/or the securing element to be closed by a closure element, in particular a ring, on the side facing towards the adapter.

This is advantageous, in particular, when the spring tongues or spring platelets are inserted into the cavities, since the closure elements and/or the rings close the cavities on the front (seen from the direction of the adapter) and, by this means, the spring tongues or the spring platelets can be prevented from dropping out. Preferably, the at least one closure element is elastic, in particular the at least one closure element consists of an elastic plastic material. As a result, the spring tongues can be pushed elastically into the cavities and can be positioned therein.

A refinement of the dispensing device according to the invention provides the dispensing device to comprise a housing with a handle, whereby the securing element is preferred to be firmly connected to the housing and the at least one propulsion body is supported as in a bearing against the housing such as to be linearly mobile.

By this means, the position of the at least one securing element with the housing is defined such that there is no need for separate storage of the securing element which simplifies the design of the dispensing device.

Moreover, the invention can provide the at least one propulsion body to surround the clamping rod, at least in regions thereof, preferably to surround it by at least 75%, particularly preferably to surround it completely.

By this means, the force can act on the clamping rod from all sides in a particularly simple manner. Moreover, this effectively prevents the dispensing device from being dismantled, such that said design is preferred.

In this context, the invention can provide the at least one propulsion body to comprise a feed-through that preferably is provided as a tube, as before.

By this means, tipping and tilting of the propulsion body on the clamping rod can be prevented.

Alternatively, the invention can just as well provide multiple axially shiftable propulsion bodies to touch against the clamping rod from different directions.

The individual propulsion body can thus be designed to be simpler. However, the assembly of the dispensing device is made more difficult and undesired dismantling of the dispensing device is made easier.

Preferred embodiments can be characterised in that the cutting edges, in particular the spring tongues or the at least one spring platelet, have a hardness of at least 45 HRC, preferably of at least 50 HRC.

This allows multiple tightening and releasing of the spring tongues on/off the clamping rod to be ensured. The term “HRC” shall be understood to refer to the Rockwell hardness according to scale C according to DIN EN ISO 6508-1.

Moreover, the invention proposes the clamping rod to consist of a plastic material, preferably of a thermosetting plastic material.

As a result, the dispensing device can be produced inexpensively essentially from plastic materials and can thus be used as a hygienic disposable article. Particularly preferably, the clamping rod consists of a plastic material from the group of phenol resins, urea resins, polyurethanes, and epoxide resins.

The invention can just as well provide the at least one securing element to be arranged on the clamping rod ahead of or behind the at least one propulsion body with respect to the propulsion direction of the clamping rod.

Preferably, the securing element is arranged on the clamping rod between the at least one propulsion body and the adapter.

Moreover, the invention proposes the spring tongues, in particular the spring platelets, to consist of spring steel.

Firstly, spring steel is sufficiently hard and stable to be able to engage a clamping rod made of plastics and not to break in the process and, secondly, is very elastic in order to provide the requisite elastic deformability when the cutting edges are to slide over the clamping rod. Moreover, the above mentioned components made of spring steel can be produced inexpensively or are commercially available inexpensively.

The objects underlying the present invention are also met by a method for propelling a clamping rod with a manually operable operating element, in which at least one propelling body touching against the clamping rod is moved in a first axial direction of the clamping rod by a manual force acting on the operating element,

whereby cutting edges of multiple spring tongues of the at least one propulsion body engage the clamping rod and thus are clamped against the clamping rod such that the clamping rod is moved in the first axial direction along with the propulsion body,
whereby, as soon as the action of the manual force on the operating element is reduced or ceases, the at least one propulsion body is pushed by a spring element in a second axial direction that is opposite to the first axial direction, whereby the operating element is restored to the starting position and whereby the cutting edges of the spring tongues of the at least one propulsion body slide over the clamping rod due to the action of a retrograde force,
and whereby a retrograde motion of the clamping rod in the second axial direction is blocked by at least one securing element touching against the clamping rod, whereby, for this purpose, cutting edges of spring tongues of the securing element engage the clamping rod due to the retrograde motion of the clamping rod and thus are clamped against the clamping rod such that the clamping rod no longer moves with respect to the at least one securing element.

According to the invention, the steps proceed chronologically in the order given.

Referring to a blockade of the motion of the clamping rod by the securing element, it needs to be understood that this is subject to a limitation in that obviously there is a minor retrograde motion of the clamping rod until the motion is fully blocked by the securing element when the cutting edges of the spring tongues of the securing element engage. The extent to which the clamping rod can move backwards depends on the retrograde force that is exerted, the number and length of the cutting edges, the support of the clamping rod, the elastic deformability of the spring tongues opposite to the propulsion direction, and the hardness of the clamping rod.

Referring to methods according to the invention, the invention proposes that the surface of the clamping rod gets plastically deformed by the cutting edges of the spring tongues of the propulsion body and of the securing element, when the cutting edges of the spring tongues of the propulsion body and of the securing element engage the clamping rod.

As a result, a stable connection of the spring tongues to the clamping rod can be attained.

The invention can also provide the cutting edges of the spring tongues of the at least one securing element to be released from the clamping rod, i.e. from the depressions in the clamping rod generated by the cutting edges, and the clamping rod to thus be moved with respect to the at least one securing element when the clamping rod moves in the first axial direction.

This ensures that the clamping rod is easy to propel. Due to the spring force of the spring tongues, the cutting edges then still touch against the clamping rod, but no longer engage the clamping rod.

Moreover, the invention proposes that the action of the manual force exerted upon manual operation of the operating element, in particular upon tilting a rocker lever as operating element, compresses the spring element.

By this means, the spring element is pre-tensioned for the subsequent restoration of the at least one propulsion body.

Preferred embodiments can provide the operation of the operating element to be repeated multiply and the clamping rod to be propelled stepwise in this context and a bone cement to be expelled stepwise in this context from a cartridge that has previously been connected to a dispensing device by means of an adapter, whereby the dispensing device comprises the adapter, the operating element, the clamping rod, the at least one propulsion body, and the securing element.

As a result, the clamping rod is propelled in stepwise manner by repeated operation of the operating element such that a single operation is associated with sufficient force for expelling the often viscous bone cement being present. The cement cartridge can be present as a single cartridge or as a cartridge system. A static mixture that may be provided on one dispensing opening of the cement cartridge might render the dispensation of the bone cement even more difficult.

Finally, the invention can provide the method to be implemented through the use or by application of a dispensing device according to the invention.

The invention is based on finding, surprisingly, that the use of a securing element and a manually driven propulsion body, which are arranged on a clamping rod such as to be mobile with respect to each other and each comprise inclined spring tongues, whereby the spring tongues engage the clamping rod upon a motion of the propulsion body or of the clamping rod and thus move the clamping rod along with the propulsion body or the securing element impedes a motion, allows a simple, but effective unidirectional propulsion of the clamping rod to be attained without there being a chance that the clamping rod is driven back by means of elastic forces of the PMMA cement cartridge. In this context, the clamping rod is, in addition, plastically deformed by the spring tongues and the clamping rod is prevented from being pushed backward by the spring tongues of the securing element engaging the clamping rod. This restricts and/or rules out any re-usability such that the use of non-sterilised dispensing devices can be prevented. Concurrently, the entire design including the clamping rod of the dispensing device can be implemented inexpensively such that the disposable product is not too expensive to manufacture.

It has also been found, surprisingly, that it is feasible through the use of the dispensing device according to the invention, despite the use of simple, inexpensive clamping rods and of inexpensive plastic parts, to dispense a polymethylmethacrylate bone cement dough without undesired retrograde motions of the dispensing plungers during the dispensation of polymethylmethacrylate bone cement dough from vacuum cementing systems. Moreover, it has been found that it is not possible to restore the dispensing device to its original condition without destroying it such that any re-use of the device after completed dispensation is excluded.

A dispensing device according to the invention can be composed of, for example, at least one manually-actuatable rocker lever (as operating element), a clamping rod, an adapter for the cement cartridge, and a housing with a handle. The dispensing device can be characterised, for example, in that

a) an axially shiftable propulsion body is arranged on the clamping rod and surrounds the clamping rod, at least in regions thereof;

b) the propulsion body possesses at least one cavity;

c) the cavity has a ring-shaped spring platelet arranged in it that possesses spring tongues on its internal surface that are oriented in the direction of the propulsion direction of the plastic clamping rod and touch against the clamping rod;

d) an elastic element (as closure of the cavity) pushes the ring-shaped spring platelet into the cavity of the propulsion body;

e) the rocker lever is arranged appropriately in the housing such that it can rotate such that, upon actuation of the rocker lever, one end of the rocker lever is being pushed onto the axially shiftable propulsion body in the propulsion direction of the plastic clamping rod;

f) a spring element pushes on the propulsion body in opposite direction to the propulsion direction of the plastic clamping;

g) the housing has a securing element connected to it that surrounds the plastic clamping rod, whereby the securing element possesses at least one wedge-shaped cavity; and

h) the cavity of the securing element has a ring-shaped spring platelet arranged in it that possesses spring tongues on its internal surface that are oriented in the direction of the propulsion direction of the plastic clamping rod and touch against the clamping rod.

The housing and the adapter for the cement cartridge preferably consist of plastic material. The rocker lever consists either of plastic material, and aluminium alloys or steel.

The rocker lever is preferably designed as a lever system in accordance with EP 0 326 551 A1. Said lever system uses a manually-actuated lever parallelogram that enables forceful actuation of the lever by the index finger or middle finger.

The feed-through of the propulsion body is preferably designed as a tube. By this means, tipping and tilting of the propulsion body on the clamping rod is excluded.

According to the invention, the spring platelets can be provided to be made from spring steel. The spring platelets are preferred to be conically-shaped, whereby the spring tongues are provided to be conical, facing inwards.

A method according to the invention for dispensing polymethylmethacrylate bone cement by means of a dispensing device according to the invention can be characterised, for example, in that
in a step a), the rocker lever is moved manually against the propulsion body that is situated in a starting position, whereby the propulsion body is clamped against the plastic clamping rod by spring platelets and moves the plastic clamping rod in the propulsion direction, whereby concurrently the spring element is being compressed,
in a step b), once the propulsion motion of the rocker lever is completed, the propulsion body is pushed back into the starting position by the compressed spring element on the clamping rod,
in a step c), the securing element is secured against moving in a direction opposite to the propulsion direction by clamping of the spring platelet or spring platelets against the plastic clamping rod, and in that steps a through c are repeated.

Preferably, the steps proceed in chronological order this context.

Further exemplary embodiments of the invention shall be illustrated in the following on the basis of five schematic figures, though without limiting the scope of the invention. In the figures:

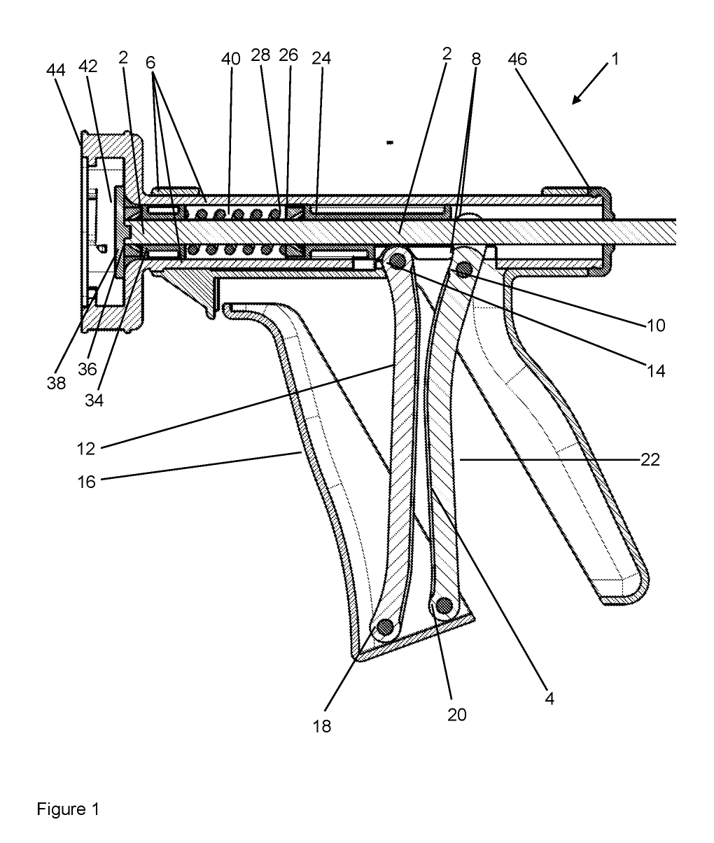

FIG. 1: shows a schematic a cross-sectional view of a dispensing device according to the invention;

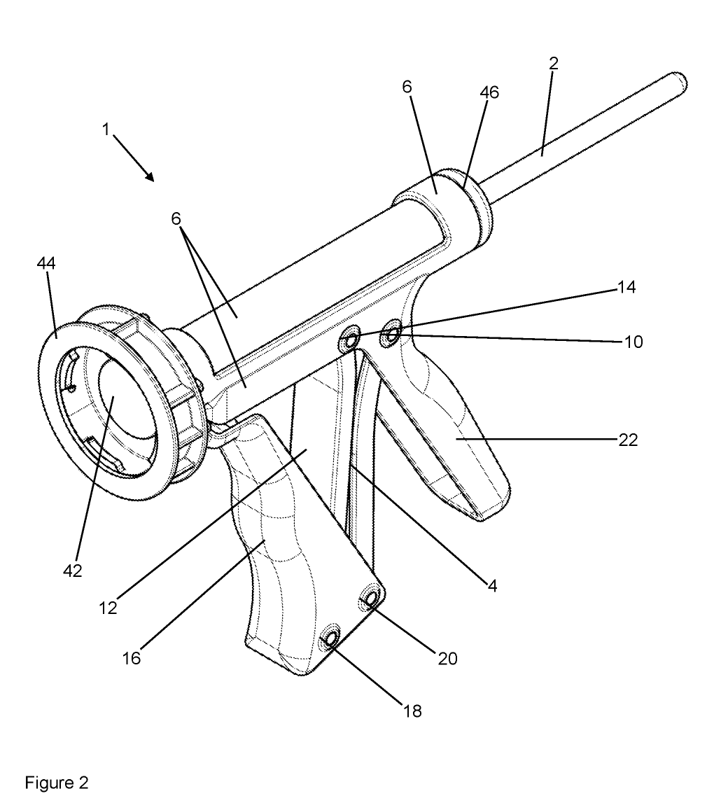

FIG. 2: shows a schematic perspective view of the dispensing device according to FIG. 1;

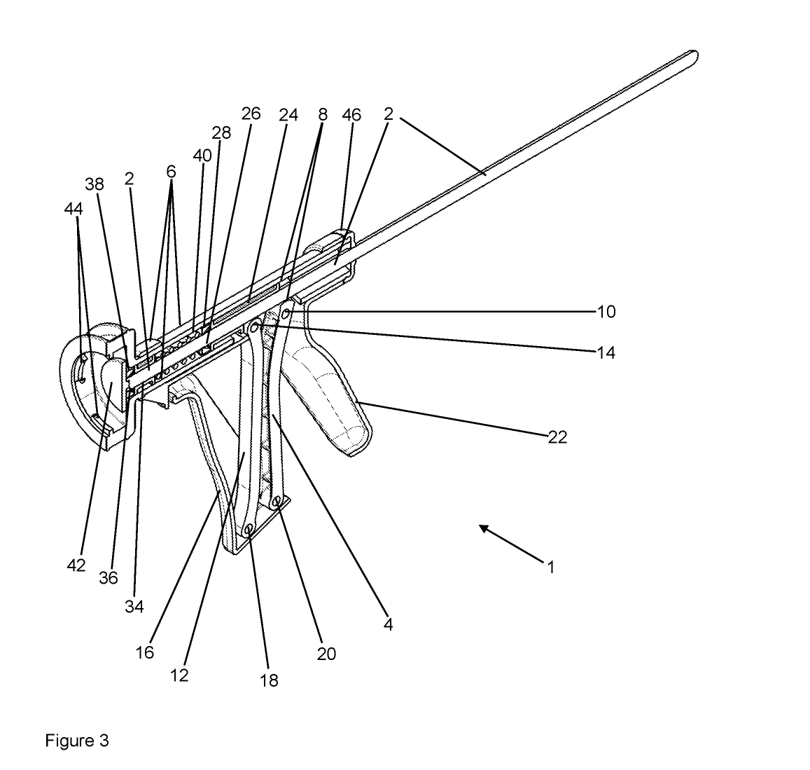

FIG. 3: shows a schematic perspective cross-sectional view of a dispensing device having a longer clamping rod;

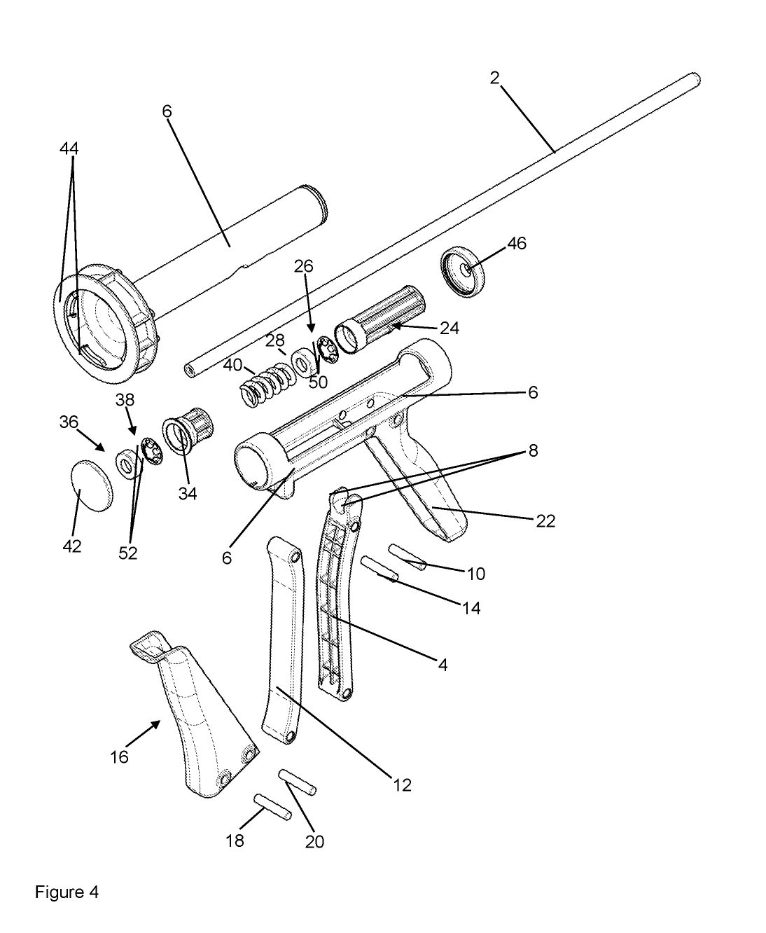

FIG. 4: shows a schematic exploded view of the dispensing device according to FIG. 3; and

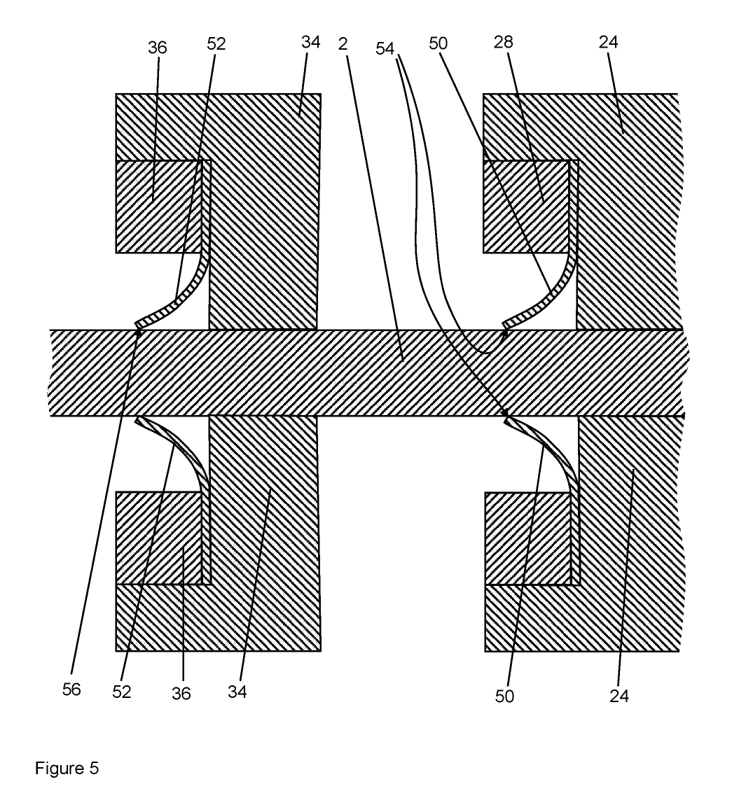

FIG. 5: shows a schematic view of a cross-section of a detail of a dispensing device according to the invention to illustrate the principle of function.

FIG. 1 shows a cross-sectional view of a dispensing device 1 with a clamping rod 2 that can be propelled forward (left in FIG. 1), whereby the sectioned areas are shown cross-hatched. FIG. 2 shows a perspective external view onto the dispensing device 1 according to FIG. 1. FIGS. 3 and 4 show a perspective cross-sectional view and an exploded view, respectively, of another dispensing device 1 that differs from the dispensing device 1 shown in FIGS. 1 and 2 only by the length of the clamping rod 2. FIG. 5 shows a schematic design of a cross-section of a detail of a dispensing device according to the invention to illustrate the principle of function. The section surfaces are shown cross-hatched in FIG. 5 as well. Identical and similar components of different dispensing devices identified by the same reference numbers in the figures.

Clamping rod 2 consists of a plastic material and can be moved by means of a rocker lever 4, as manually actuatable operating element 4, that is made of a stable plastic material or, alternatively, of steel or aluminium. The dispensing device 1 is largely surrounded by a multi-part housing 6 made of plastics such that the internal design of the dispensing device 1 is not exposed. The housing parts 6 can be manufactured as injection moulding parts. The rocker lever 4 terminates in a pivoting head 8 by means of which the motion of the rocker lever 4 is transmitted into the inside of the housing 6 of the dispensing device 1. For this purpose, the rocker lever 4 is connected to the housing 6 by means of an axle 10. When the rocker lever 4 is rotated about the axle 10 (counter-clockwise in the top view onto the cross-section according to FIG. 1 and the perspective view according to FIGS. 2 and 3), the head 8 of the rocker lever 4 is pushed forward at great force due to the leverage effect (towards the left in FIGS. 1, 2, and 3).

The rocker lever 4 is operated by moving a strut 12 that is connected to the housing 6 by means of an axis 14 and to a trigger 16 by means of an axis 18. The rocker lever 4 is also connected to the trigger 16, namely by means of an axle 20. Due to this design, the trigger 16 can be moved parallel to a handle 22. Being a part of the housing 6, the handle 22 is made of plastics. Due to the design involving the rocker lever 4 and the strut 12 as well as their connections/axles 10, 14, 18, 20 to the housing 6 and the trigger 16, the full height of the trigger 16 can be used to exert a pressure onto the rocker lever 4. By this means, the rocker lever 4 can be operated with the full force of the entire hand in particular including the force of the index finger and middle finger, whereby the handle 22 is being held by the same hand. Accordingly, the entire dispensing device 1 is easy to hold and operate with one hand. A design of this type is described in detail in EP 0 326 551 A1 as well. The axles 10, 14, 18, 20 can be manufactured from plastics, since there can be no significant wear and tear considering the few strokes of the rocker lever 4.

The head 8 of the rocker lever 4 is used to propel a propulsion body 24 forward (shifting it towards the left in FIGS. 1, 2, 3, and 5). The propulsion body 24 surrounds the clamping rod 2 and comprises, on the inside, a recess that forms a cavity together with the clamping rod 2. The cavity has a spring platelet 26 arranged in it that comprises multiple spring tongues 50 made of spring steel. The spring tongues 50 are provided as clamping bodies and touch, in springlike manner, against the clamping rod 2 by means of cutting edges 54. The cavity, which is bounded by the propulsion body 24 and the clamping rod 2 as well as a closure 28 on the front side (on the left in FIGS. 1, 2, 3, and 5), has, along with the spring platelet 26, a multitude of spring tongues 50 arranged in it, whereby the spring tongues 50 surround the clamping rod 2.

When the head 8 of the rocker lever 4 pushes onto the propulsion body 24 due to a rotation about the axle 10, the cutting edges 54 of the spring tongues 50 are pressed against the clamping rod 2. Due to the orientation of the spring tongues 50, the cutting edges 54 cut into and/or engage the clamping rod 2. As a result, the spring tongues 50 and the propulsion body 24 and the clamping rod 2 become lodged and/or wedged against each other. Since the contact surface (contact points) of the cutting edges 54 on the clamping rod 2 is small, a very high pressure is generated at the contact surfaces that is sufficient to plastically deform the clamping rod 2. For this purpose, the hardness of the cutting edges 54 or of the spring tongues 50 or of the entire spring platelet 26 made of spring steel is higher than the hardness of the clamping rod 2 made of plastics. The incised cutting edges 54 then block a propulsion of the propulsion body 24 on the clamping rod 2 by means of the spring tongues 50 and/or by means of the spring platelet 26.

The front side of the dispensing device 1 (on the left in FIGS. 1, 3, and 5) has a securing element 34 provided on it, which is identical in design to the propulsion body 24, whereby the securing element 34 is firmly connected to the housing 6, whereas the propulsion body 24 is supported as in a bearing to be mobile in the housing 6.

The securing element 34 surrounds the clamping rod 2 and comprises, on the inside, a recess that forms a cavity together with the clamping rod 2. A spring platelet 36 made of spring steel is arranged in the cavity as clamping body. Multiple spring tongues 52 of the spring platelet 36 touch, by cutting edges 56, against the clamping rod 2 on all sides. The spring platelet 36 is enclosed in and attached to the cavity, which is bounded by the securing element 34 and the clamping rod 2 as well as a closure 38 on the front side (on the left in FIGS. 1, 2, 3, and 5). The spring platelets 26, 36 shown in the exploded view according to FIG. 4 each comprise six inward-facing spring tongues 50, 52, which each are arranged in pairs opposite from each other and, in the assembled state, are all inclined in the same direction with respect to the propulsion direction of the clamping rod 2.

When the clamping rod 2 with the rocker lever 4 and the propulsion body 24 is propelled forward, the clamping rod 2 slides through the securing element 34 since the cutting edges 56 can easily slide over the clamping rod 2 during this motion, since this motion permits an elastic deformation of the spring tongues 52. Concurrently, an elastic spring 40 that is arranged between the propulsion body 24 and the securing element 34 in the housing 6 is being tensioned and/or compressed elastically.

When the force acting on the rocker lever 4 lessens or ceases, the tensioned spring 40 pushes the propulsion body 24 in the opposite direction towards the back (towards the right in FIGS. 1, 2, 3, and 5). During this motion, the cutting edges 54 on the spring tongues 50 of the spring platelet 26 of the propulsion body 24 can slide on the clamping rod 2 due to the inclination of the spring tongues 50 with respect to the clamping rod 2, since the spring tongues 50 can be elastically deformed easily during this motion. As a result, the propulsion body 24 can be shifted on the clamping rod 2 by means of the spring 40.

Concurrently, is not feasible to push the clamping rod 2 back towards the rear into the housing 6 (towards the right in FIGS. 1, 2, 3, and 5), since this motion is blocked by the cutting edges 56 due to the inclination of the spring tongues 52 of the securing element 34 with respect to the clamping rod 2. Accordingly, if the extrusion of bone cement from a cartridge (not shown) is associated with elastic forces exerting a counter-pressure onto the clamping rod 2, the clamping rod 2 cannot be pushed back into the housing 6.

The front side of the clamping rod 2 has a punch 42 attached to it that is intended for propelling a feed plunger (not shown) of a cement cartridge (not shown). An adapter 44 with a bayonet closure for connecting a cement cartridge is situated on the front side of the dispensing device 1. The cement cartridge (not shown) can be attached to the adapter 44, whereby the bottom of the attached cement cartridge contains the feed plunger that can be pushed into the cement cartridge by means of the punch 42. When the clamping rod 2 is being propelled, the punch 42 pushes the feed plunger into the cement cartridge, whereby the cartridge content (for example a medical PMMA bone cement) is pushed from the cement cartridge through a cartridge opening that is situated opposite from the feed plunger.

The dispensing device 1 is closed on the back by a cap 46. The cap 46 comprises a feed-through for the clamping rod 2 and can be considered to be a part of the housing 6.

The features of the invention disclosed in the preceding description and in the claims, figures, and exemplary embodiments, can be essential for the implementation of the various embodiments of the invention both alone and in any combination.

### LIST OF REFERENCE NUMBERS

1 Dispensing device
2 Clamping rod
4 Rocker lever/operating element
6 Housing
8 Head of the rocker lever
10 Axis
12 Strut
14 Axis
16 Trigger
18 Axis
20 Axis
22 Handle
24 Propulsion body
26 Spring platelet
28 Closure
34 Securing element
36 Spring platelet
38 Closure
40 Spring
42 Punch
44 Adapter/bayonet connector
46 Cap
50 Spring tongue
52 Spring tongue
54 Cutting edge
56 Cutting edge

1. A dispensing device for cement cartridges for polymethylmethacrylate bone cements comprising:
at least one manually actuatable operating element;
an adapter for a cement cartridge; and
a clamping rod that can be propelled in the direction of the adapter,
wherein at least one propulsion body is arranged to be touching against the clamping rod and is shiftable in the dispensing device in axial direction with respect to an axle of the clamping rod,
wherein the at least one propulsion body comprises multiple elastic spring tongues, wherein the spring tongues each comprise a cutting edge, whereby the spring tongues touch against the clamping rod by means of the cutting edge, wherein the cutting edges of the spring tongues or the entire spring tongues are harder than the clamping rod, and whereby the spring tongues of the propulsion body are inclined appropriately with respect to the clamping rod such that, upon a motion of the propulsion body in the propulsion direction of the clamping rod, the cutting edges of the propulsion body engage the clamping rod, and such that, upon a motion of the propulsion body opposite to the propulsion direction of the clamping rod, the spring tongues of the propulsion body can be elastically deformed appropriately such that the cutting edges slide over the clamping rod,
wherein a spring element pushes with an elastic force, at least for part of the time, onto the at least one propulsion body opposite to the propulsion direction of the clamping rod,
wherein a force can be exerted on the at least one propulsion body in the propulsion direction of the clamping rod by means of the operating element, wherein the propulsion body and the clamping rod can be propelled forward relative to the adapter against the elastic force of the spring element by means of said force,
wherein a securing element, against which the propulsion body is supported such as to be mobile, touches against the clamping rod, wherein the securing element comprises multiple elastic spring tongues, whereby the spring tongues each comprise a cutting edge, whereby the spring tongues touch against the clamping rod by means of the cutting edge, wherein at least the cutting edges of the spring tongues or the entire spring tongues are harder than the clamping rod, and
wherein the spring tongues are inclined appropriately with respect to the clamping rod such that, upon a motion of the clamping rod opposite to the propulsion direction, the cutting edges of the securing element engage the clamping rod, and such that, upon a motion of the clamping rod in the propulsion direction, the spring tongues of the securing element can be elastically deformed appropriately such that the cutting edges slide over the clamping rod.

2. The dispensing device according to claim 1, wherein the at least one propulsion body is linearly mobile in axial direction with respect to the clamping rod and with respect to the adapter and/or in that the securing element is connected to the dispensing device such as to be immobile with respect to the adapter.

3. The dispensing device according to claim 1, wherein the cutting edges of the spring tongues of the securing element appropriately engage the clamping rod during a motion of the clamping rod opposite to the propulsion direction such that any further motion of the clamping rod opposite to the propulsion direction is prevented.

4. The dispensing device according to claim 1, wherein the cutting edges of the spring tongues of the at least one propulsion body engage the clamping rod appropriately such that the clamping rod moves along with the at least one propulsion body in propulsion direction.

5. The dispensing device according to claim 1, wherein the elastic spring tongues of the at least one propulsion body and of the securing element are inclined appropriately such that the cutting edges are oriented in the propulsion direction and the further parts of the spring tongues extend from the cutting edges opposite to the propulsion direction, whereby the spring tongues are inclined at an angle between 88° and 20° with respect to the axle of the clamping rod or at an angle between 80° and 70° with respect to the axle of the clamping rod.

6. The dispensing device according to claim 1, wherein the elastic spring tongues of the at least one propulsion body and of the securing element touch against the clamping rod appropriately such that the cutting edges are pushed onto the clamping rod by the elastic force of the elastically deformed spring tongues.

7. The dispensing device according to claim 1, wherein the at least one manually actuatable operating element is a manually actuated rocker lever, wherein the rocker lever is supported as in a bearing such that it is rotatable with respect to the clamping rod, such that, upon actuation of the rocker lever, an end of the rocker lever pushes onto the at least one axially shiftable propulsion body in propulsion direction of the clamping rod.

8. The dispensing device according to claim 1, wherein the elastic spring tongues are arranged on the internal surface of at least one ring-shaped spring platelet, wherein the at least one spring platelet surrounds the clamping rod completely or by more than 50%.

9. The dispensing device according to claim 1, wherein the at least one propulsion body touching against the clamping rod and the clamping rod jointly form at least one cavity and/or that the securing element touching against the clamping rod and the clamping rod jointly form at least one cavity, whereby the spring tongues are arranged, in particular the at least one ring-shaped spring platelet is arranged, in the cavity or cavities.

10. The dispensing device according to claim 9, wherein the cavity formed by the propulsion body and/or the securing element is closed by a ring on the side facing towards the adapter.

11. The dispensing device according to claim 1, further comprising:
a housing with a handle, wherein the securing element is firmly connectable to the housing, and the at least one propulsion body is supported as in a bearing against the housing such as to be linearly mobile.

12. The dispensing device according to claim 1, wherein the at least one propulsion body surrounds the clamping rod, at least 75% thereof or surrounds it completely.

13. The dispensing device according to claim 12, wherein the at least one propulsion body comprises a feed-through that preferably is provided as a tube.

14. The dispensing device according to claim 1, further comprising:
multiple axially shiftable propulsion bodies touching against the clamping rod from different directions.

15. The dispensing device according to claim 1, wherein the spring tongues or the at least one spring platelet have a hardness of at least 45 HRC.

16. The dispensing device according to claim 1, wherein the clamping rod consists of a thermosetting plastic material.

17. The dispensing device according to claim 1, wherein the at least one securing element is arranged on the clamping rod ahead of or behind the at least one propulsion body with respect to the propulsion direction of the clamping rod.

18. The dispensing device according to claim 1, wherein the spring tongues consist of spring steel.

19. A method for propelling a clamping rod with a manually operable operating element, in which at least one propulsion body touching against the clamping rod is movable in a first axial direction of the clamping rod by a manual force acting on the operating element, the method comprising:
engaging cutting edges of multiple spring tongues of the at least one propulsion body with the clamping rod and thus are clampable against the clamping rod such that the clamping rod is movable in the first axial direction along with the propulsion body,
wherein, as the manual force acting on the operating element is reduced or ceases, the at least one propulsion body is pushed by a spring element into a second axial direction that is opposite to the first axial direction, wherein the operating element is restored to the starting position and wherein the cutting edges of the spring tongues of the at least one propulsion body so slide over the clamping rod due to the action of a retrograde force, and
wherein a retrograde motion of the clamping rod in the second axial direction is blockable by at least one securing element touching against the clamping rod, wherein, cutting edges of spring tongues of the securing element engage the clamping rod due to the retrograde motion of the clamping rod and thus are clampable against the clamping rod such that the clamping rod no longer moves with respect to the at least one securing element.

20. The method according to claim 19, wherein the surface of the clamping rod plastically deforms by the cutting edges of the spring tongues of the propulsion body and of the securing element, when the cutting edges of the spring tongues of the propulsion body and of the securing element engage the clamping rod.

21. The method according to claim 19, wherein the cutting edges of the spring tongues of the securing element are released from the depressions in the clamping rod generated by the cutting edges, and the clamping rod is thus movable with respect to the at least one securing element, when the clamping rod moves in the first axial direction.

22. The method according to claim 19, wherein the action of the manual force exerted upon tilting a rocker lever as operating element compresses the spring element.

23. The method according to claim 19, wherein the operation of the operating element is multiply repeated, the clamping rod is propelled stepwise in this context, and a bone cement is expelled stepwise in this context from a cement cartridge that has previously been connected to a dispensing device by means of an adapter, wherein the dispensing device comprises the adapter, the operating element, the clamping rod, the at least one propulsion body, and the securing element.

24. The method according to claim 19, wherein the method is performed via the so dispensing device according to claim 1.
